SEQUENCE LISTING (2 sequences)

SEQ ID NO: 1 - protein, 118 aa, synthetic construct
ESGGGSVQAGGSLRLSCAASGYTYSTYCMAWFRQALGKEREGVAGIDSDGSTTYADSVKGRFTISKDNAKDTLYLQMDSLKPEDTAMYYCAALSPRCDFDRSSRVTNWGQGTQVTVSS

SEQ ID NO: 2 - protein, 119 aa, synthetic construct
ESGGGSVQAGGSLRLSCVVSGYTSRHCMGWFRQAPGKEREGVAALYPATGSAFYGDSVKGRFTISQDNAKNTLYLQMNSLKPDDTALYYCAVESTSLCAARTVPSFKFRGQGTQVTVSS

(19) United States
(12) Patent Application Publication
(10) Pub. No.: US 2026/0243759 A1
(43) Pub. Date: Aug. 20, 2026

(54) PHOTONIC CRYSTAL BIOCHIP AND PROTEIN DETECTION METHOD THEREOF

(71) Applicant: BEIJING INSTITUTE OF HEART, LUNG AND BLOOD VESSEL DISEASES, Beijing (CN)

(72) Inventors: Jie DU, Beijing (CN); Yuan WANG, Beijing (CN); Xue WANG, Beijing (CN); Bingjie XUE, Beijing (CN)

(73) Assignee: BEIJING INSTITUTE OF HEART, LUNG AND BLOOD VESSEL DISEASES, Beijing (CN)

(21) Appl. No.: 19/164,224

(22) PCT Filed: Dec. 28, 2022
(86) PCT No.: PCT/CN2022/142780

§ 371 Date: Sep. 11, 2025

(30) Foreign Application Priority Data
Jun. 8, 2022 (CN) 202210637489.8

(51) Int. Cl. G01N 33/543; G01N 21/64; G01N 33/545; G01N 33/68

(52) U.S. Cl. CPC G01N 33/54313; G01N 21/64; G01N 33/545; G01N 33/6869; G01N 2333/7155

(57) ABSTRACT

A photonic crystal biochip and a protein detection method thereof are provided. The technical problem to be solved is how to obtain a photonic crystal biochip capable of rapid and/or simple and/or sensitive protein detection. The photonic crystal biochip includes a reaction substrate layer, where the reaction substrate layer is provided with at least one stable capture zone and at least one labile detection zone; the stable capture zone includes a photonic crystal array and at least one capture antibody; and the labile detection zone includes a photonic crystal array and at least one detection antibody. The disclosure enables rapid, simple, and microscale protein detection.

### TECHNICAL FIELD

The disclosure belongs to the field of testing or analyzing materials by virtue of the chemical or physical properties of tested materials, and specifically relates to a photonic crystal biochip and a protein detection method thereof.

### BACKGROUND TECHNOLOGY

ST2, also known as human growth stimulation expressed gene 2, is a member of the IL-1 receptor family and primarily exists in two isoforms: transmembrane ST2L (membrane-bound receptor form, ST2 receptor) and soluble sST2 (soluble ST2). ST2L serves as the functional receptor for IL-33, and the IL-33/ST2L signaling pathway has demonstrated cardioprotective effects, whereas sST2 binding to IL-33 downregulates this pathway. sST2 is predominantly produced by vascular endothelial cells under stress or injury, with elevated concentrations confirmed in patients with acute myocardial infarction, acute heart failure, and particularly in aortic dissection, exhibiting high specificity and sensitivity. Quantitative determination of circulating sST2 in humans enables auxiliary differential diagnosis and prognostic evaluation for cardiovascular diseases. Despite its clinical potential, sST2-related detection assays are not yet widely implemented, and universally applicable sST2 detection methods remain lacking as an emerging biomarker.

In detection methodologies involving signal transduction, amplification of detectable signals can significantly enhance detection sensitivity. Photonic crystals find extensive applications in fluorescent sensor technologies. By introducing photonic crystal structures into conventional fluorescence detection systems, photonic crystal fluorescent sensors leverage fluorescence enhancement effects to amplify fluorescent signals, thereby improving detection sensitivity and reducing limits of detection. Photonic crystals, characterized by their periodic structures, enhance electromagnetic field interactions with fluorescent molecules when localized within photonic crystal domains, amplifying spontaneous emission fluorescence signals for enhanced detectability. Although fluorescence labeling represents a critical approach for biomolecular detection, challenges such as low analyte concentrations, high background noise, and microsample volumes persist, compromising detection reliability and accuracy. The integration of photonic crystals into detectors enhances fluorescence signals, elevating device sensitivity for ultra-sensitive detection and analysis of DNA, proteins, and other biomolecules, holding significant implications for medical diagnostics. Patent CN102161284A discloses a method for fabricating responsive patterned colloidal photonic crystal films via inkjet printing technology. US200510011219.2 details a preparation method for poly (methyl methacrylate)-acrylic acid-styrene latex spheres. Patent CN113912729A describes a nanobody preparation method.

### DISCLOSURE OF THE INVENTION

The technical problem to be solved by the disclosure is how to obtain a photonic crystal biochip capable of rapid and/or simple and/or sensitive protein detection.

The disclosure provides a photonic crystal biochip including a reaction substrate layer, wherein the reaction substrate layer is provided with at least one stable capture zone and at least one labile detection zone; the stable capture zone includes a photonic crystal array and at least one capture antibody; the labile detection zone includes a photonic crystal array and at least one detection antibody labeled with a fluorescent group.

The fluorescent group is selected from at least one of FAM, VIC, HEX, TRT, CY3, CY5, ROX, JOE, FITC, TET, NED, TAMRA, LC RED460, LC RED705, Quasar705, or Texas Red.

The capture antibody is an antibody capable of capturing a target protein to be detected, the detection antibody is an antibody capable of binding to the target protein and labeled with a fluorescent group, and the fluorescent signal may be a Cy5 group.

The reaction substrate layer is a polymer sheet or polymer film.

The reaction substrate layer is fabricated from polyester, polystyrene, polymethacrylic acid, polypropylene, or polyvinyl chloride.

The photonic crystals are poly(methyl methacrylate)-acrylic acid-styrene latex spheres.

The poly(methyl methacrylate)-acrylic acid-styrene latex spheres have a particle size (diameter) of 300 nm.

The biochip is a photonic crystal biochip for detecting soluble ST2 protein, wherein the capture antibody is a soluble ST2 protein capture antibody; the detection antibody is a soluble ST2 protein detection antibody.

The soluble ST2 protein capture antibody is designated as capture antibody NbA, which is antibody 1A9 disclosed in patent CN113912729A.

The soluble ST2 protein detection antibody is designated as detection antibody Cy5-NbB, which is antibody 2B10 disclosed in patent CN113912729A conjugated with a Cy5 group.

The disclosure further provides a method for detecting a target protein, including applying a test sample to the biochip and performing fluorescence detection, wherein a fluorescent signal indicates the presence of the target protein, while the absence of a fluorescent signal indicates its absence.

The target protein may be soluble ST2 protein, in which case the capture antibody is a soluble ST2 protein capture antibody, and the detection antibody is a soluble ST2 protein detection antibody.

The soluble ST2 protein capture antibody is designated as capture antibody NbA, which may be single-domain antibody 1A9 having the amino acid sequence set forth in SEQ ID NO: 1.

The soluble ST2 protein detection antibody is designated as detection antibody Cy5-NbB, which may be single-domain antibody 2B10 conjugated with a Cy5 group, where the single-domain antibody 2B10 has the amino acid sequence set forth in SEQ ID NO: 2.

The application of the photonic crystal biochip in detecting or supporting the detection of soluble ST2 protein shall also fall within the scope of protection of the disclosure.

The disclosure further provides a preparation method for a photonic crystal biochip, including the following steps:
1) uniformly coating an adhesive layer on the surface of a reaction substrate layer to obtain a substrate;
2) inkjet-printing a photonic crystal array onto the substrate from step 1 using a liquid containing photonic crystal microspheres as ink, to obtain a substrate bearing the photonic crystal array;
3) drying the substrate bearing the photonic crystal array from step 2, followed by heat curing;
4) upon completion of curing, activating the photonic crystal array, introducing capture antibodies to bind with the photonic crystal array, and forming a stable capture zone;
5) performing a blocking step using bovine serum albumin (BSA); and
6) immobilizing detection antibodies at the periphery of the photonic crystal array to form a labile detection zone.

The capture antibody is an antibody capable of capturing the target protein to be detected, and the detection antibody is an antibody capable of binding to the target protein and labeled with a fluorescent signal, which may be a Cy5 group.

The liquid containing photonic crystal microspheres is formulated from 919 parts by volume of poly(methyl methacrylate-co-acrylic acid-co-styrene) latex spheres, 80 parts by volume of ethylene glycol, and 1 part by volume of surfactant; the poly(methyl methacrylate-co-acrylic acid-co-styrene) latex spheres have a particle size of 300 nm; the heating and curing conditions are 110° C. for 10 min.

The capture antibody is an ST2 protein capture antibody; the detection antibody is an ST2 protein detection antibody.

The soluble ST2 protein capture antibody is designated as capture antibody NbA, which may be single-domain antibody 1A9 having the amino acid sequence set forth in SEQ ID NO: 1.

The soluble ST2 protein detection antibody is designated as detection antibody Cy5-NbB, which may be single-domain antibody 2B10 conjugated with a Cy5 group, wherein the amino acid sequence of single-domain antibody 2B10 is set forth in SEQ ID NO: 2.

The photonic crystal biochip of the disclosure, by incorporating photonic crystals, enables rapid, simple, and microscale protein detection. When applied to soluble ST2 protein detection, the disclosure achieves rapid, simple, and microscale quantification of soluble ST2 protein.

### DESCRIPTION OF THE DRAWINGS

FIG. 1 illustrates the basic workflow for detecting soluble ST2 protein using a photonic crystal biochip fabricated via green printing technology.

FIG. 2 presents microscope and scanning electron microscope (SEM) images of several photonic crystal biochips prepared in Example 1, where (a) demonstrates fluorescence enhancement effects of photonic crystal microspheres with varying particle sizes, and (b) shows SEM images of photonic crystal microspheres with different diameters.

FIG. 3 compares detection results between the proposed method and conventional enzyme-linked immunosorbent assay (ELISA) for soluble ST2 protein at different concentration gradients in Example 3. Specifically, (a) shows fluorescence images of detection chips with photonic crystals across standard curve concentrations; (b) presents fluorescence intensity statistics for detection chips without photonic crystals (R²=0.96); (c) displays fluorescence intensity statistics for detection chips with photonic crystals (R²=0.975); and (d) illustrates time-dependent fluorescence intensity changes for detection chips incorporating photonic crystals.

FIG. 4 compares the overall linear trends between photonic crystal biochip-based detection (as per the disclosure) and ELISA for 15 clinical samples.

FIG. 5 shows fluorescence intensity comparisons of Cy5-NbB on photonic crystal arrays with varying particle sizes in Example 5.

FIG. 6 contrasts the performance of biochips fabricated using IgG antibodies versus nanobodies (as per the disclosure) coupled with PC300 photonic crystal arrays in Example 6.

### BEST WAY TO IMPLEMENT THE INVENTION

The present disclosure is further described in detail below with reference to specific embodiments. The embodiments provided are solely for the purpose of illustrating the disclosure and do not limit its scope. These embodiments serve as a guide for further improvements by ordinary technicians in the technical field and do not in any way restrict the scope of the present disclosure.

Unless otherwise specified, the experimental methods described in the following embodiments are conventional methods carried out in accordance with the techniques or conditions described in the literature within the field or following the product specifications. All materials, reagents, etc., used in the following embodiments are commercially available unless otherwise specified, and “room temperature” as mentioned in the embodiments refers to 25° C.

Experimental Samples: The following examples utilize 15 patients who voluntarily participated and sought treatment at Beijing Anzhen Hospital between October 2021 and November 2021.

The “green printing method” referenced in the subsequent embodiments refers to the method for fabricating responsive patterned colloidal photonic crystal films via inkjet printing technology as described in Chinese patent CN102161284A. The capture antibody NbA used in the examples is single-domain antibody 1A9 with the amino acid sequence set forth in SEQ ID NO: 1, while the detection antibody Cy5-NbB is single-domain antibody 2B10 conjugated with a Cy5 group, where the amino acid sequence of single-domain antibody 2B10 is set forth in SEQ ID NO: 2. These antibodies may be prepared according to the methods disclosed in patent CN113912729A or commissioned from Nanjing Rong Jie Kang Biotechnology Co., Ltd. The amino acid sequence of 1A9 is as follows:

ESGGGSVQAGGSLRLSCAASGYTYSTYCMAWFRQALGKEREGVAGI
DSDGSTTYADSVKGRFTISKDNAKDTLYLQMDSLKPEDTAMYYCAA
LSPRCDFDRSSRVTNWGQGTQVTVSS.

The amino acid sequence of 2B10 is as follows:

ESGGGSVQAGGSLRLSCVVSGYTSRHCMGWFRQAPGKEREGVAALY
PATGSAFYGDSVKGRFTISQDNAKNTLYLQMNSLKPDDTALYYCAV
ESTSLCAARTVPSFKFRGQGTQVTVSS.

The PBS referenced in the subsequent examples is Zhongshan Golden Bridge ZLI-9061, the FBS is Gibco 0025, the healthy human EDTA-anticoagulated serum is sourced from the Health Examination Center of Beijing Anzhen Hospital, and the recombinant protein sST2 is ACROBiosystems il-H5229. The poly(methyl methacrylate-co-acrylic acid-co-styrene) latex spheres used in the examples are prepared via the method disclosed in Chinese patent 200510011219.2, yielding an emulsion containing monodisperse poly(styrene-methyl methacrylate-acrylic acid) triblock copolymer latex particles at 50° C. The prepared poly (methyl methacrylate-co-acrylic acid-co-styrene) latex spheres have particle sizes of 280 nm, 300 nm, 310 nm, 320 nm, 360 nm, and 370 nm, respectively.

### Example 1: Selection of Photonic Crystal Microsphere Particle Size

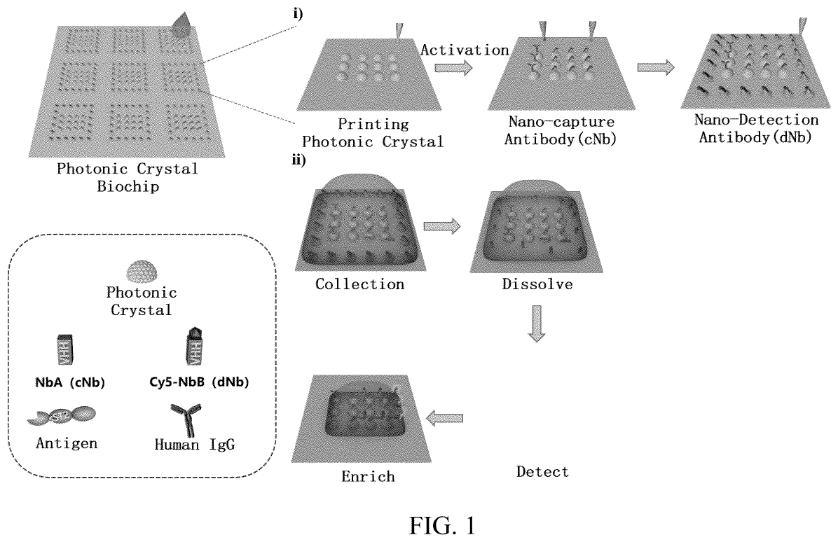

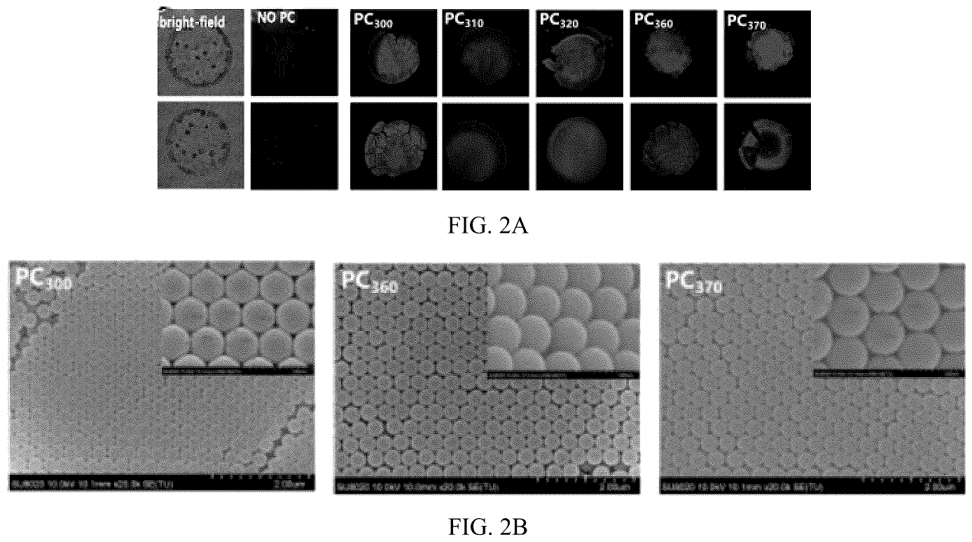

Photonic crystal arrays were prepared following the method illustrated in steps a-c of FIG. 1, as detailed below.
a) A layer of adhesive (polyvinyl alcohol aqueous adhesive) was uniformly coated on the surface of a PET substrate to immobilize subsequently printed photonic crystal microspheres.
b) Using a liquid containing photonic crystal microspheres as “photonic ink,” inkjet printing was performed on the substrate from step a) using a SONOguide instrument. Each 5×5 matrix array had a side length not exceeding 1 mm. The photonic ink included a mixed solution of poly (methyl methacrylate-co-acrylic acid-co-styrene) latex spheres, ethylene glycol, and a surfactant (BYK-3455 silicone surfactant from BYK, Germany) in a volume ratio of 919:80:1. This formulation adjusted the drying rate of the photonic ink post-printing to ensure ordered assembly of the microspheres. The poly(methyl methacrylate-co-acrylic acid-co-styrene) latex spheres were prepared via the method disclosed in Chinese patent ZL200510011219.2, yielding an emulsion containing monodisperse poly(styrene-methyl methacrylate-acrylic acid) triblock copolymer latex particles with nominal diameters of 300 nm, 310 nm, 320 nm, 360 nm, and 370 nm.
c) After drying, the biochip was cured at 110° C. for 10 min in an oven.

Using the aforementioned protocol, photonic crystal arrays were fabricated with poly (methyl methacrylate-co-acrylic acid-co-styrene) latex spheres of diameters 300 nm, 310 nm, 320 nm, 360 nm, and 370 nm, yielding PC₃₀₀, PC₃₁₀, PC₃₂₀, PC₃₆₀, and PC₃₇₀ arrays (corresponding to FIG. 2).

Optical microscopy (FIG. 2a) revealed angle-dependent red photonic crystal colors in the printed arrays. Scanning electron microscopy (FIG. 2b) demonstrated ordered and densely packed microsphere arrangements. Upon spotting Cy5 dye onto the photonic crystal biochip surface and drying, confocal microscopy quantified fluorescence enhancement effects. Reflectance spectroscopy confirmed that the 300 nm photonic crystal microspheres exhibited optimal bandgap alignment with Cy5 emission.

### Example 2: Preparation of Photonic Crystal Chip for Capturing Soluble ST2 Protein

A photonic crystal chip capable of capturing soluble ST2 protein was fabricated using a Nordson EFD dispenser and the green printing method. The printing parameters included a 5×5 dot matrix with a diameter of approximately 240 μm, a pitch of 0.5 mm between dots, and an inter-matrix spacing of 7 mm, as illustrated in FIG. 1. The detailed procedure is as follows:
1) Photonic crystal array-300 (PC300) was prepared according to the method described in Example 1.
2) The carboxyl groups on the surface of photonic crystal microspheres in the PC300 array were activated using Sulfo-NHS (SIGMA24510)/EDC (SIGMA22981) ester. Nanobody NbA (capture antibody) was introduced to undergo dehydration condensation with the amino groups provided by the nanobody, enabling covalent conjugation of NbA to the photonic crystal array surface.
3) Blocking treatment was performed with 5% BSA at room temperature to minimize non-specific binding during subsequent detection.
4) The fluorescently labeled detection antibody Cy5-NbB was non-covalently immobilized on the chip surface via inkjet printing, arranged at the periphery of the photonic crystal array. The dried antibody arrays (at 25° C.) retained activity under dry storage conditions, yielding the photonic crystal detection chip.

During detection, 20 μL of test solution was applied to the chip. The peripheral Cy5-NbB detection antibody array dissolved, enabling recognition and binding of the target analyte. As solvent evaporation progressed, the droplet volume decreased. Due to the hydrophilic nature of the photonic crystal array compared to the PET substrate, the droplet retracted toward the center, facilitating capture of the analyte-NbB complex by the central photonic crystal array-bound NbA (capture antibody). Angle-dependent fluorescence enhancement from the photonic crystal structure amplified the fluorescent signal. All reagents required for detection were pre-printed on the mm-scale chip surface, significantly streamlining the operational workflow.

### Example 3

Gradient concentrations of sST2 solutions (0.01, 0.1, 1, 10, 100, and 1000 ng/ml) were prepared using PBS, FBS, or healthy human EDTA-anticoagulated serum as solvents to serve as test samples.

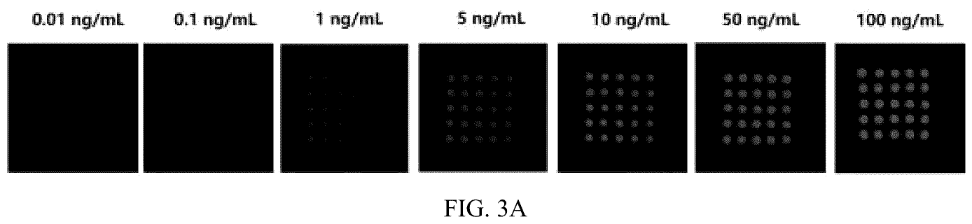

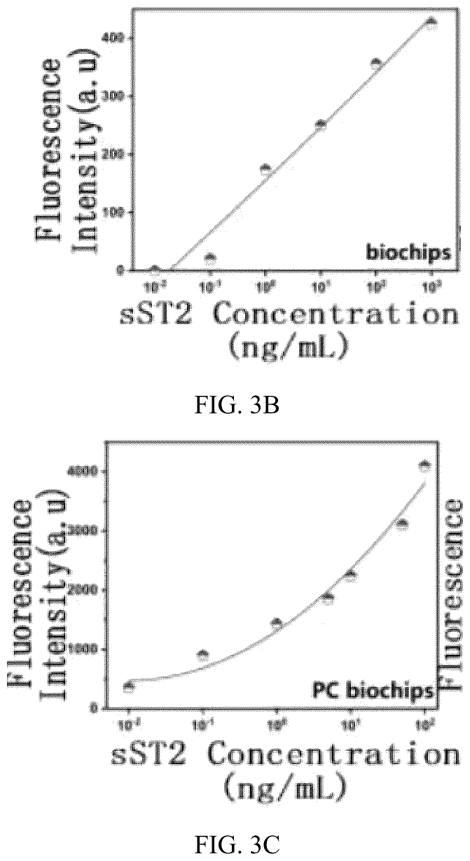

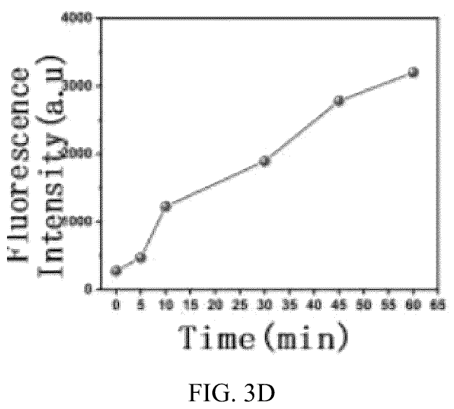

The aforementioned samples were subjected to performance evaluation using two methods: (1) photonic crystal-based fluorescence-linked immunosorbent assay (PC-FLISA) as described in Example 2, and (2) conventional fluorescence-linked immunosorbent assay (FLISA). Detection results are presented in FIG. 3a. The conventional FLISA protocol was conducted following the procedures outlined in the literature titled “Performance of coumarin-derived dendrimer-based fluorescence-linked immunosorbent assay (FLISA) to detect malaria antigen.” As shown in FIG. 3a, the PC-FLISA method demonstrated significantly higher fluorescence intensity than conventional FLISA at equivalent analyte concentrations.

Confocal laser scanning microscopy was employed to capture detection results, and fluorescence intensity data were statistically analyzed to determine linear ranges and limits of detection (LOD). Results are illustrated in FIGS. 3b-3d. The PC-FLISA assay achieved a detectable antigen concentration as low as 0.01 ng/ml with consistent signal enhancement.

### Example 4

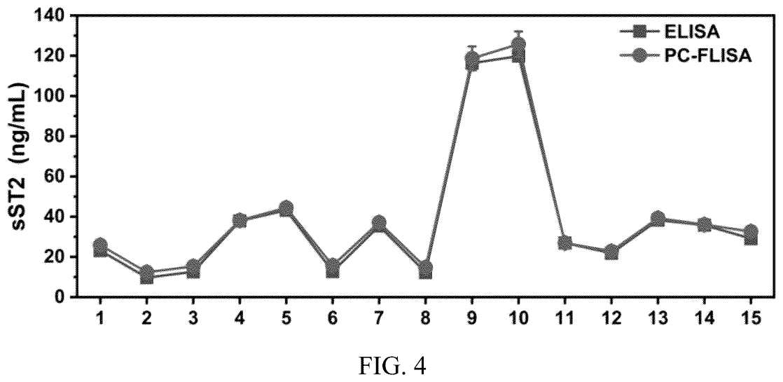

Blood samples from 15 cardiovascular disease patients were diluted 10-fold with PBS to prepare test solutions. These samples were analyzed using both the photonic crystal detection chip of the present disclosure and conventional ELISA, with results shown in FIG. 4. The conventional ELISA protocol was performed following the methodology described in the literature: “Enzyme immunoassay (EIA)/enzyme-linked immunosorbent assay (ELISA)” (Lequin RM, Clin Chem. 2005 December; 51 (12): 2415-8. doi: 10.1373/clinchem.2005.051532).

As demonstrated in FIG. 4, the overall linear trend of detection results obtained with the inventive PC-FLISA method was comparable to that of the gold-standard ELISA. Replicate measurements confirmed the stability and consistency of PC-FLISA results across identical samples under repeated testing conditions.

### Example 5: Comparative Analysis of Nanoparticles with Varying Diameters

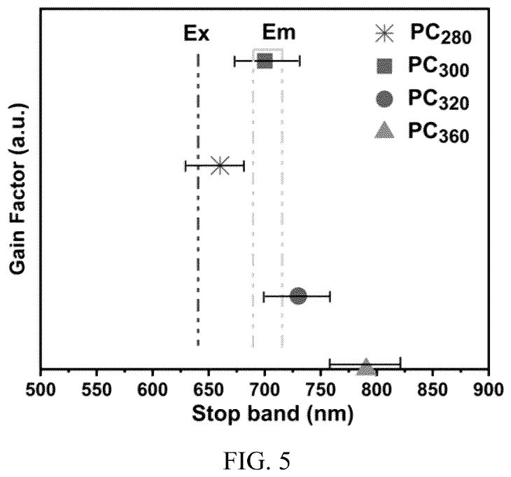

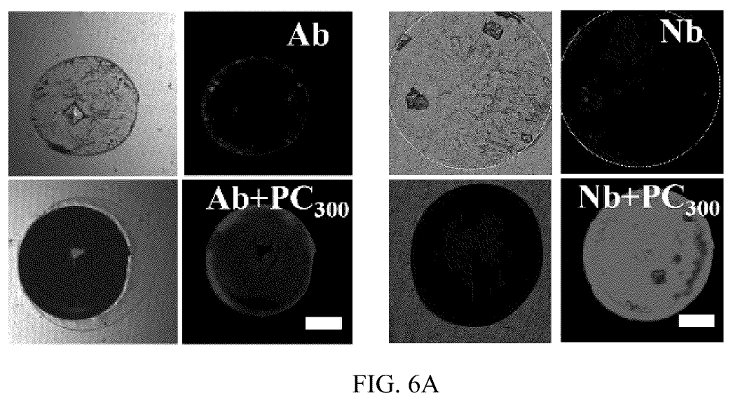

Photonic crystal arrays PC280, PC300, PC320, and PC360 were fabricated using poly (methyl methacrylate-co-acrylic acid-co-styrene) latex spheres with nominal diameters of 280 nm, 300 nm, 320 nm, and 360 nm, respectively, following the protocol described in Example 1. Fluorescence intensity of Cy5-NbB (0.5 μL, 450 ng/ml) immobilized on these arrays was compared, with results presented in FIG. 5. PC300 demonstrated the highest emission enhancement (60.3-fold) due to alignment between its photonic bandgap edge and the emission wavelength of Cy5-NbB, significantly amplifying spontaneous emission of dyes localized within the photonic crystal matrix voids. While PC280 exhibited enhanced excitation efficiency through bandgap edge matching with the excitation wavelength, its photonic bandgap overlapped with Cy5-NbB emission, resulting in partial emission suppression and only 39.8-fold enhancement. PC320 and PC360, with bandgaps distant from both excitation and emission wavelengths of Cy5-NbB, provided moderate 10.8-fold and 1.5-fold enhancements, respectively, attributed to scattering effects and increased surface area. These findings confirm that PC300 is optimal for maximizing Cy5-NbB fluorescence signal enhancement.

### Example 6: Comparative Data of IgG Antibody (Ab) and Nanobody (Nb)

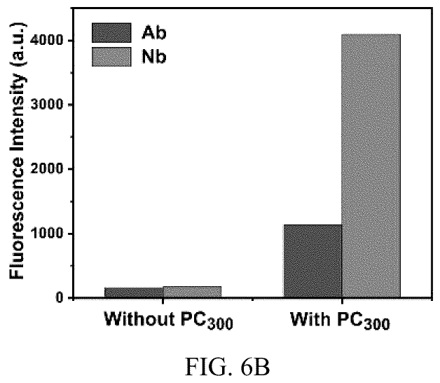

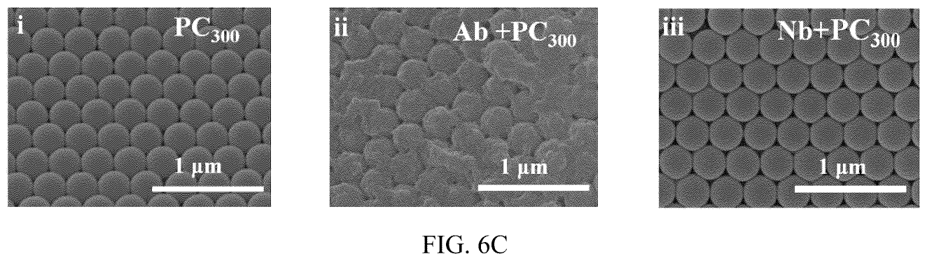

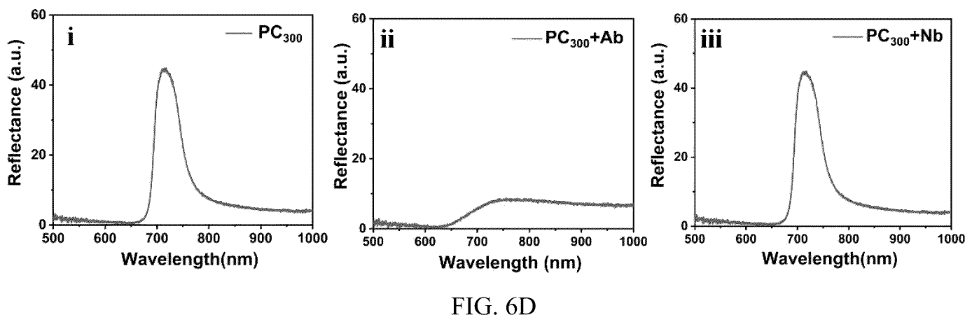

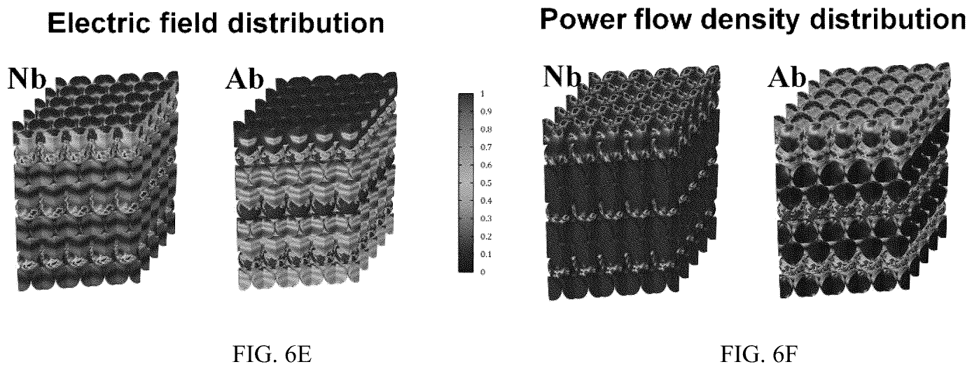

Photonic crystal chips were prepared using PC₃₀₀ or blank substrates following the method described in Example 2, with Cy5-labeled IgG antibody and Cy5-labeled nanobody (Nb) respectively, and their performance was evaluated. Detection results are presented in FIG. 6.

### FIG. 6:

a) Optical bright-field and fluorescence images of Cy5-labeled IgG antibody (0.5 μL, 10 μg/mL) and Cy5-labeled nanobody (0.5 μL, 10 μg/mL) immobilized on photonic crystal array PC300 and blank substrates. Labeling: Ab=photonic crystal chip prepared with Cy5-IgG and blank substrate; Nb=photonic crystal chip prepared with Cy5-nanobody and blank substrate; Ab+PC₃₀₀=photonic crystal chip prepared with Cy5-IgG and PC₃₀₀ substrate; Nb+PC₃₀₀=photonic crystal chip prepared with Cy5-nanobody and PC₃₀₀ substrate.
b) Fluorescence intensity analysis of Cy5-IgG and Cy5-nanobody on PC₃₀₀ arrays versus blank substrates. Notably, fluorescence enhancement of Cy5-nanobody on PC₃₀₀ substrates significantly exceeded that of Cy5-IgG. Labeling: Ab=Cy5-IgG antibody used; Nb=Cy5-nanobody used; Without PC₃₀₀=blank substrate used; With PC₃₀₀=PC₃₀₀ substrate used.
c) Scanning electron microscopy (SEM) images: (i) Colloidal spheres assembled in PC₃₀₀ dots; (ii) IgG antibodies (Ab) immobilized on PC₃₀₀ surface; (iii) Nanobodies (Nb) immobilized on PC₃₀₀ surface. IgG antibodies disrupted the ordered assembly of PC₃₀₀ colloidal spheres, resulting in a non-smooth surface morphology. Labeling: PC₃₀₀=photonic crystal array; Ab+PC₃₀₀=Cy5-IgG/PC₃₀₀ chip; Nb+PC₃₀₀=Cy5-nanobody/PC₃₀₀ chip.
d) Reflectance spectra of bare PC₃₀₀, PC₃₀₀ coupled with IgG antibodies (Ab+PC₃₀₀), and PC₃₀₀ coupled with nanobodies (Nb+PC₃₀₀). IgG antibody conjugation caused significant reflectance suppression, reducing fluorescence enhancement efficiency. Labeling: PC₃₀₀=photonic crystal array; Ab+PC₃₀₀=Cy5-IgG/PC₃₀₀ chip; Nb+PC₃₀₀=Cy5-nanobody/PC₃₀₀ chip.
e) Simulated electric field distributions for nanobody (Nb) and IgG antibody (Ab) conjugated to the photonic crystal array. Labeling: Ab=Cy5-IgG conjugated to PC₃₀₀; Nb=Cy5-nanobody conjugated to PC₃₀₀.
f) Simulated power flux density distributions for IgG antibody (Ab) and nanobody (Nb) within the photonic crystal array. Labeling: Ab=Cy5-IgG conjugated to PC₃₀₀; Nb=Cy5-nanobody conjugated to PC₃₀₀.

The aforementioned results demonstrate that conventional antibodies such as IgM or IgG, due to their larger molecular structures, pose challenges when conjugated to nanoscale photonic crystal arrays. Specifically, the conjugation process becomes operationally complex, and the bulky antibodies significantly compromise the surface smoothness of the photonic arrays, thereby attenuating their inherent fluorescence signal amplification capability. In contrast, the nanoscale single-domain antibodies (nanobodies) employed in this disclosure exhibit minimal impact on photonic array surface morphology post-conjugation, preserving optimal conditions for fluorescence signal enhancement and consequently improving detection sensitivity and accuracy. Furthermore, the compact size of nanobodies allows for significantly higher antibody density per unit area, thereby enhancing detection efficiency.

The foregoing description provides a detailed elaboration of the present disclosure. For those skilled in the art, modifications and improvements may be implemented across a broad spectrum of equivalent parameters, concentrations, and conditions without departing from the spirit and scope of this disclosure, and without requiring undue experimentation. While specific embodiments have been disclosed, it is understood that further refinements to the disclosure are contemplated. In essence, this application intends to encompass all modifications, applications, and adaptations of the disclosure that adhere to its underlying principles, including alterations achieved through conventional techniques known in the art, even if such modifications extend beyond the explicitly disclosed embodiments. Fundamental features as defined by the accompanying claims shall govern the scope of protection.

### INDUSTRIAL APPLICATION

The photonic crystal biochip of the present disclosure enables rapid, simple, and micro-volume protein detection by incorporating photonic crystal technology. Specifically, when applied to soluble ST2 protein detection, this disclosure achieves high-speed, user-friendly, and ultra-sensitive quantification.

The photonic crystal array employs 300 nm poly(methyl methacrylate-co-acrylic acid-co-styrene) latex spheres in combination with Cy5 fluorophore groups, establishing optimal spectral matching between the photonic structure and fluorescent signals. This configuration amplifies detected signals by 100-fold, significantly enhancing detection precision and sensitivity.

Conventional antibodies (e.g., IgM/IgG) possess large molecular structures that complicate conjugation to nanoscale photonic crystal arrays. Such bulky antibodies disrupt array surface smoothness, compromising inherent fluorescence amplification capabilities. In contrast, the disclosure utilizes compact single-domain antibodies (typically nanoscale), which minimally impact photonic array surface morphology upon conjugation. This preservation of structural integrity maintains optimal conditions for fluorescence signal enhancement, thereby improving detection sensitivity and accuracy. Furthermore, the nanoscale dimensions of single-domain antibodies enable significantly higher antibody density per unit area, enhancing overall detection efficiency.

What is claimed is:

1. A photonic crystal biochip, comprising a reaction substrate layer, wherein the reaction substrate layer is provided with at least one stable capture zone and at least one unstable detection zone; the at least one stable capture zone comprises a first photonic crystal array and at least one capture antibody; and the at least one unstable detection zone comprises a second photonic crystal array and at least one detection antibody labeled with a fluorophore.

2. The photonic crystal biochip according to claim 1, wherein the reaction substrate layer is a polymer sheet or a polymer film.

3. The photonic crystal biochip according to claim 2, wherein the reaction substrate layer is made of polyester, polystyrene, polymethacrylic acid, polypropylene, or polyvinyl chloride.

4. The photonic crystal biochip according to claim 1, wherein photonic crystals in the photonic crystal biochip are poly(methyl methacrylate-co-acrylic acid-co-styrene) latex spheres.

5. The photonic crystal biochip according to claim 4, wherein the poly(methyl methacrylate-co-acrylic acid-co-styrene) latex spheres have a particle diameter of 300 nm.

6. The photonic crystal biochip according to claim 5, wherein the photonic crystal biochip is configured to detect a soluble human growth stimulation expressed gene 2 (ST2) protein, wherein the at least one capture antibody is a soluble ST2 protein capture antibody and the at least one detection antibody is a soluble ST2 protein detection antibody.

7. A method for detecting a target protein, comprising: applying a test sample to the photonic crystal biochip according to claim 1, performing fluorescence detection, and determining a presence of the target protein in the test sample based on fluorescence detection results.

8. A method for preparing a photonic crystal biochip, comprising the following steps:
1) uniformly coating a layer of adhesive on a surface of a reaction substrate layer to obtain a substrate;
2) printing a photonic crystal array on the substrate using a liquid containing photonic crystal microspheres as ink to obtain a substrate with the photonic crystal array;
3) drying the substrate with the photonic crystal array and heat-curing;
4) after curing, activating the photonic crystal array, adding a capture antibody to bind with the photonic crystal array, and forming a stable capture zone;
5) blocking with bovine serum albumin (BSA); and
6) immobilizing a detection antibody at a periphery of the photonic crystal array to form an unstable detection zone.

9. The method according to claim 8, wherein the liquid containing the photonic crystal microspheres is prepared from 919 volume parts of poly (methyl methacrylate-co-acrylic acid-co-styrene) latex spheres, 80 volume parts of ethylene glycol, and 1 volume part of a surfactant; the poly(methyl methacrylate-co-acrylic acid-co-styrene) latex spheres have a particle diameter of 300 nm; and heat-curing conditions are 110° C. for 10 min.

10. The method according to claim 8, wherein the capture antibody is a soluble ST2 protein capture antibody and the detection antibody is a soluble ST2 protein detection antibody.

11. The method according to claim 7, wherein in the photonic crystal biochip, the reaction substrate layer is a polymer sheet or a polymer film.

12. The method according to claim 11, wherein in the photonic crystal biochip, the reaction substrate layer is made of polyester, polystyrene, polymethacrylic acid, polypropylene, or polyvinyl chloride.

13. The method according to claim 7, wherein photonic crystals in the photonic crystal biochip are poly(methyl methacrylate-co-acrylic acid-co-styrene) latex spheres.

14. The method according to claim 13, wherein in the photonic crystal biochip, the poly (methyl methacrylate-co-acrylic acid-co-styrene) latex spheres have a particle diameter of 300 nm.

15. The method according to claim 14, wherein the photonic crystal biochip is configured to detect a soluble ST2 protein, wherein the at least one capture antibody is a soluble ST2 protein capture antibody and the at least one detection antibody is a soluble ST2 protein detection antibody.
